(19) United States
(12) Patent Application Publication
(10) Pub. No.: US 2023/0274840 A1
(43) Pub. Date: Aug. 31, 2023

(54) DISEASE RISK EVALUATION METHOD, DISEASE RISK EVALUATION DEVICE, AND DISEASE RISK EVALUATION PROGRAM

(71) Applicant: THINKMEDICAL INC., Tokyo (JP)

(72) Inventor: Takeshi OKANOUE, Tokyo (JP)

(21) Appl. No.: 18/006,873

(22) PCT Filed: Jul. 27, 2021
(86) PCT No.: PCT/JP2021/027776

§ 371 Date: Jan. 26, 2023

(30) Foreign Application Priority Data
Jul. 28, 2020 (JP) 2020-127010

(51) Int. Cl. G16H 50/30; G16H 10/40; G16B 20/00

(52) U.S. Cl. CPC G16H 50/30; G16B 20/00; G16H 10/40

(57) ABSTRACT

A fatty liver disease risk evaluation device can generate, through use of an estimation model generating unit, an estimation model for estimating the risk level of fatty liver disease by machine learning using attribute data such as sex and age, physical finding data such as height and weight, and doctor diagnostic results in addition to blood test data as an estimation model. In the fatty liver disease risk estimation device, a data acquisition unit acquires not only blood test data of a subject, but also attribute data such as sex and age, and physical finding data such as height and weight. In the fatty liver disease risk evaluation device, a risk level inference unit furthermore evaluates the risk of fatty liver disease based on the estimation model generated by the estimation model generating unit and the subject data acquired by the data acquisition unit.

### RELATED APPLICATIONS

The present application is a National Phase of International Application Number PCT/JP2021/027776 filed Jul. 27, 2021, which claims the benefit of priority from Japanese Patent Application No. 2020-127010, filed on Jul. 28, 2020.

### TECHNICAL FIELD

The present invention relates to a disease risk evaluation method, a disease risk evaluation device, and a risk evaluation program, such as fatty liver disease.

### BACKGROUND OF THE INVENTION

A method as disclosed in Patent Document 1 has been known for providing a non-invasive method for determining the pathology of liver disease, such as non-alcohol fatty liver disease. The method in the Patent Document 1 includes measuring the amount of marker molecules contained in blood collected from a subject, determining an index value from a normalized score calculated based on the amount of the marker molecules in the same group, and determining that the subject may be suffering from NASH when the index value is greater than the reference value.

### CITATION LIST

### Patent Literature

Patent Document 1: WO 2016/163539

### SUMMARY OF INVENTION

The method as disclosed in the above patent document is promising for assessing and evaluating the risk of fatty liver disease. However, it is desirable to further improve the accuracy to be used as an alternative to a definite diagnosis by liver biopsy, for example.

The purpose of the present invention is to provide a disease risk evaluation method, a disease risk evaluation device, and a disease risk evaluation program, which are possible to more accurately evaluate the risk of liver disease and the like.

### Solution to Problem

(1) A disease risk evaluation method according to the present invention to solve the above-described problem;
an estimated model generation step of generating an estimated model for estimating a disease risk by machine learning using (a) an attribute data, (b) physical finding data, (c) a blood examination data, and (d) a diagnosis result,
a data acquisition step of acquiring (a) an attribute data of a subject, (b) physical finding data of the subject, and (c) a blood examination data of the subject,
a risk level evaluation step of inputting the data acquired in the data acquisition step into the estimated model generated in the estimated model generation step and obtaining an inferred disease risk of the subject.

In the estimation model generation step, in addition to usual blood test data, an attribute data such as gender and age, physical findings data such as height and weight, and physician’s diagnosis are used as a learning model to generate an estimation model for estimating the degree of risk of various diseases through machine learning. In the present invention, by using the estimation model, and by using subject’s attribution such as gender and age, subject’s physical findings such as height and weight in addition to subject’s blood data, it enables more accurate evaluation of the disease risks than the method of conventional technology.

(2) A disease risk evaluation device according to the present invention, which is provided to solve the aforementioned problem, comprises:
an estimated model generation circuitry generating an estimated model for estimating a disease risk by machine learning using (a) an attribute data, (b) physical finding data, (c) a blood examination data, and (d) a diagnosis result data,
a data acquisition circuitry acquiring (a) an attribute data of a subject, (b) physical finding data of the subject, and (c) a blood examination data of the subject,
a risk level evaluation circuitry inputting the data acquired in the data acquisition step into the estimated model generated in the estimated model generation step, and obtaining an inferred disease risk of the subject.

The disease risk evaluation device can generate an estimation model for estimating the degree of risk of various diseases by machine learning, using attribute data such as gender and age, physical findings data such as height and weight, and physician’s diagnosis results as learning models, in addition to blood test data, by the estimation model generation circuitry. In addition, the data acquisition circuitry acquires not only the subject’s blood test data, but also the subject’s attribute data such as gender and age, and the subject’s physical findings data such as height and weight. Furthermore, based on the estimation model generated by the estimation model generation circuitry and the subject’s data acquired by the data acquisition circuitry, the risk degree evaluation circuitry can evaluate the risk level of various diseases. Therefore, according to the disease risk evaluation device in the present invention, the risk of various diseases can be more accurately evaluated compared to prior art methods.

(3) A disease risk assessment program of the present invention that is provided to solve the aforementioned problem is for causing a computer to implement the functions of the disease risk assessment device according to (2) above

According to such an arrangement, the risk of various diseases can be evaluated by using a computer, and the evaluation accuracy and the processing speed can be improved.

### Advantageous Effects of Invention

According to the present invention, it is possible to provide a disease risk evaluation method, a liver disease risk evaluation device, and a liver disease risk evaluation program capable of more accurately evaluating a disease risk such as a fatty liver disease.

### BRIEF DESCRIPTION OF THE DRAWINGS

FIG. 1 illustrates a NASH-Scope structure (AI system for NAFLD screening) for training and verification.

FIG. 2 illustrates the structure of Fibro-Scope for sharpening and verification (AI system for identifying the stage of liver fibrosis in NASH).

FIGS. 3-1 and FIGS. 3-2 are examples of a progression NASH diagnosed with NASH-Scope and Fibro-Scope.

FIG. 4A, FIG. 4B and FIG. 4C are diagrams showing a comparative example of the diagnostic accuracy of Fibro-Scope and associated NIT 6;

FIG. 5 shows Table 1, which is referenced in the form for carrying out the invention.

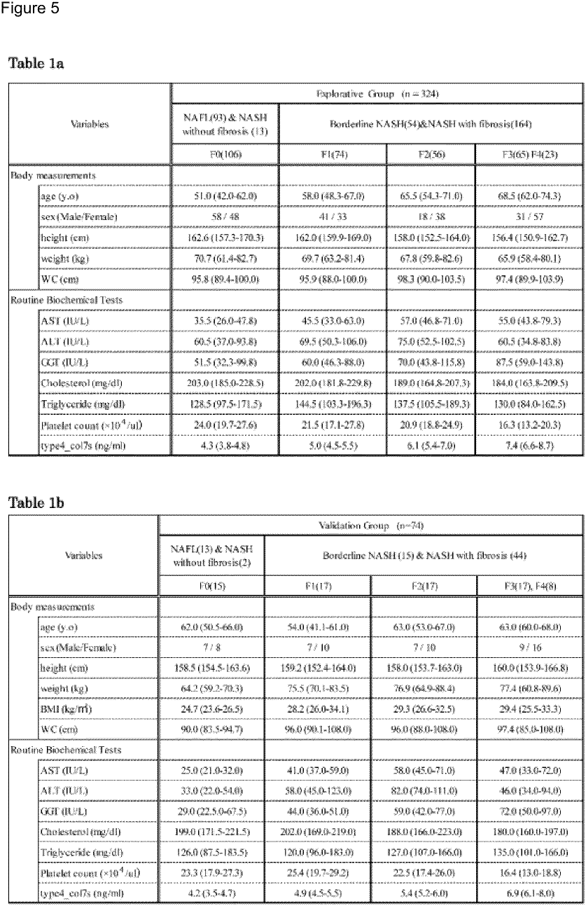

FIG. 6 shows Table 2, which is referenced in the form for carrying out the invention.

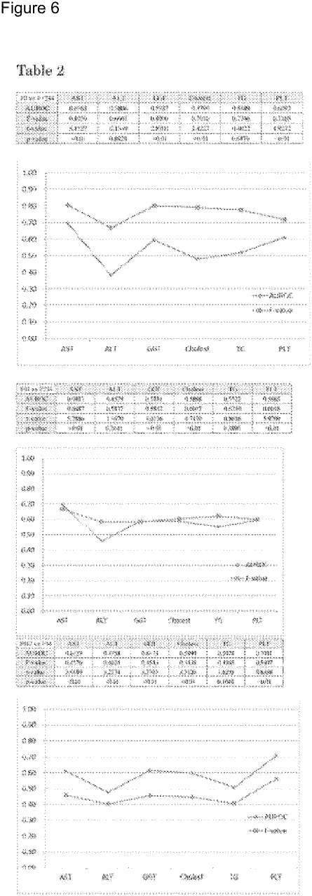

FIG. 7 shows Table 3, which is referenced in the form for carrying out the invention.

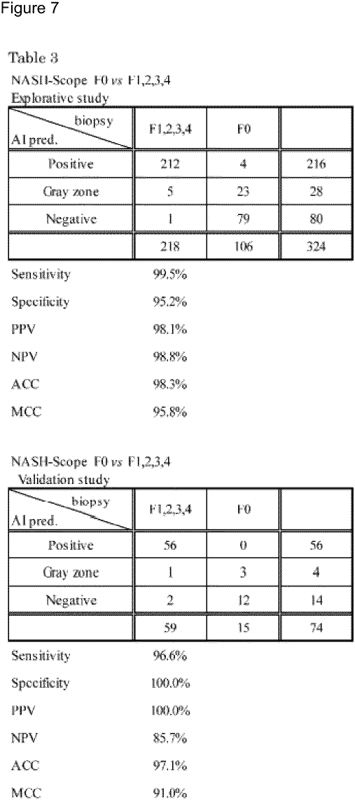

FIG. 8 shows Table 4, which is referenced in the form for carrying out the invention.

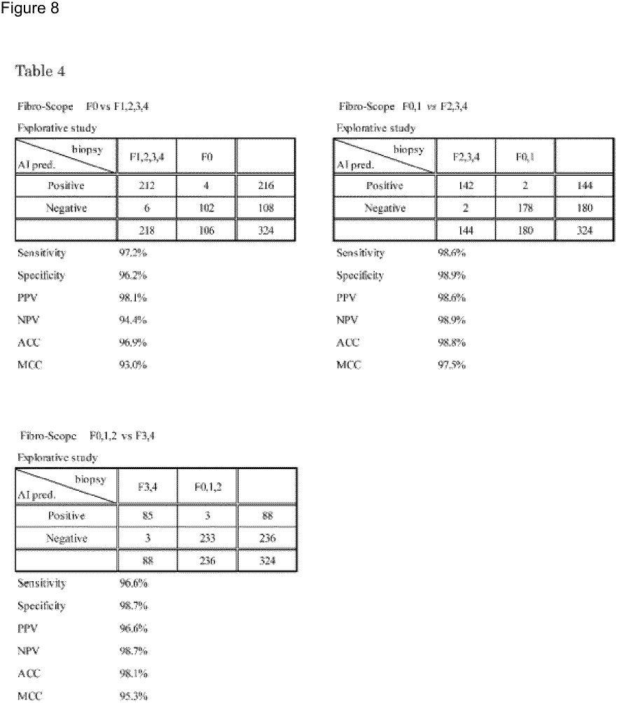

FIG. 9 shows a portion of Table 5 referenced in the Detailed Description.

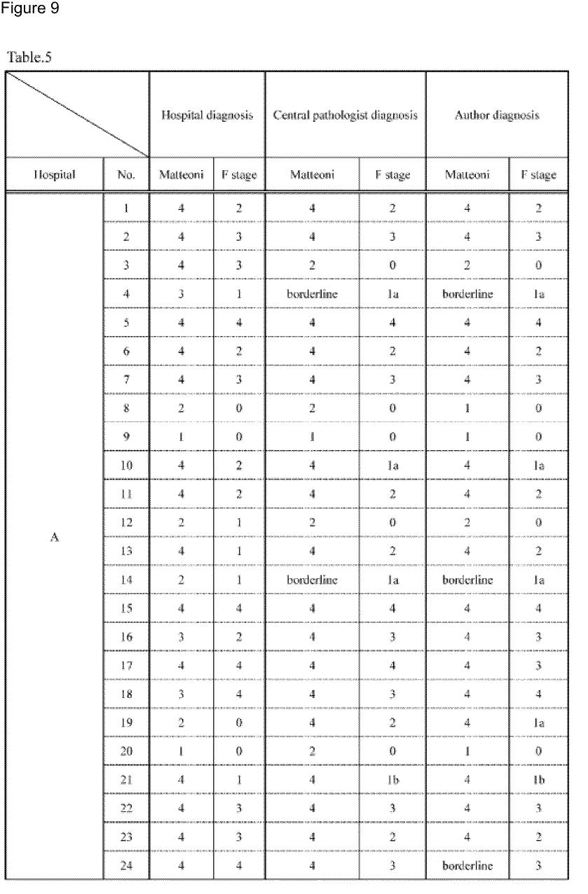

FIG. 10 shows a portion of Table 5 referenced in the Detailed Description.

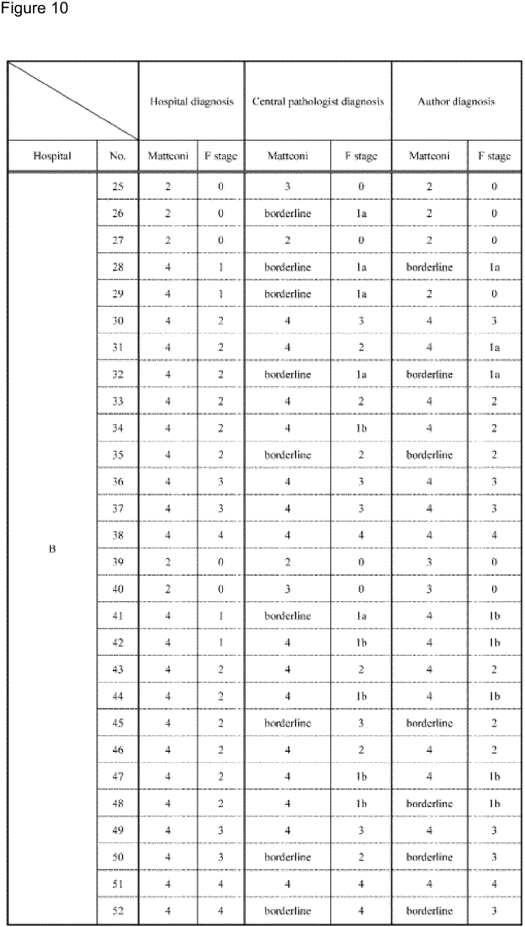

FIG. 11 shows a portion of Table 5 referenced in the Detailed Description.

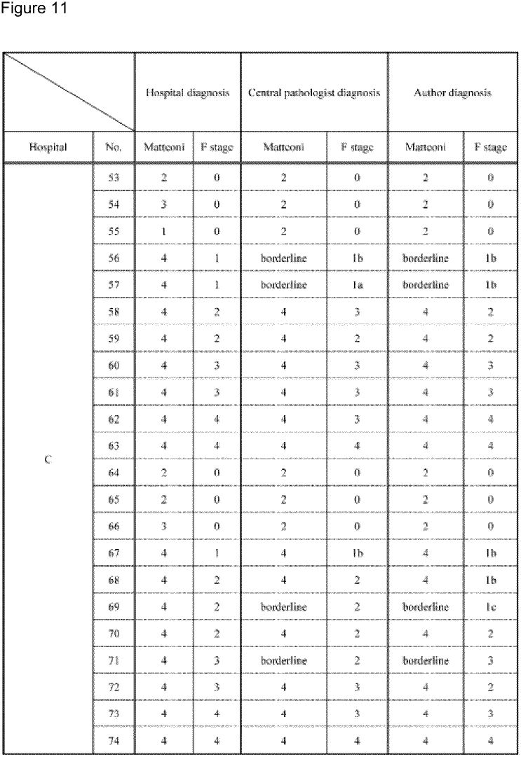

FIG. 12 shows Table 6, which is referenced in the form for carrying out the invention.

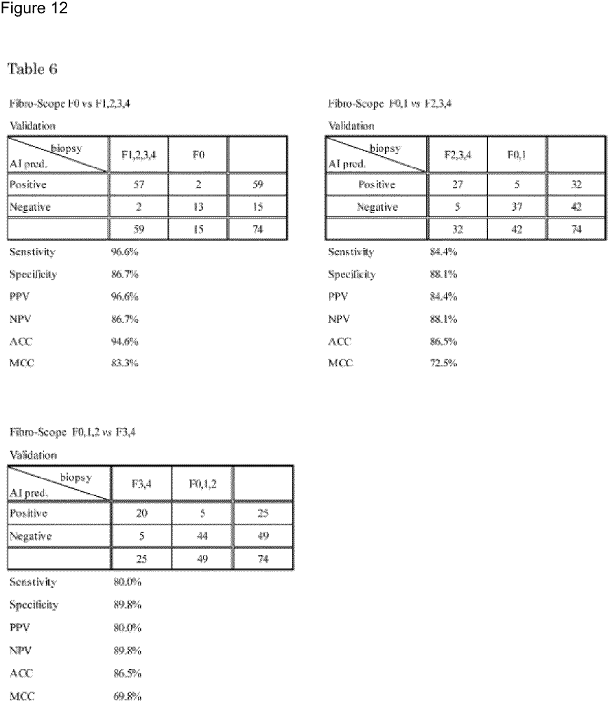

FIG. 13 shows a Table 7 referred to in a form for carrying out the invention.

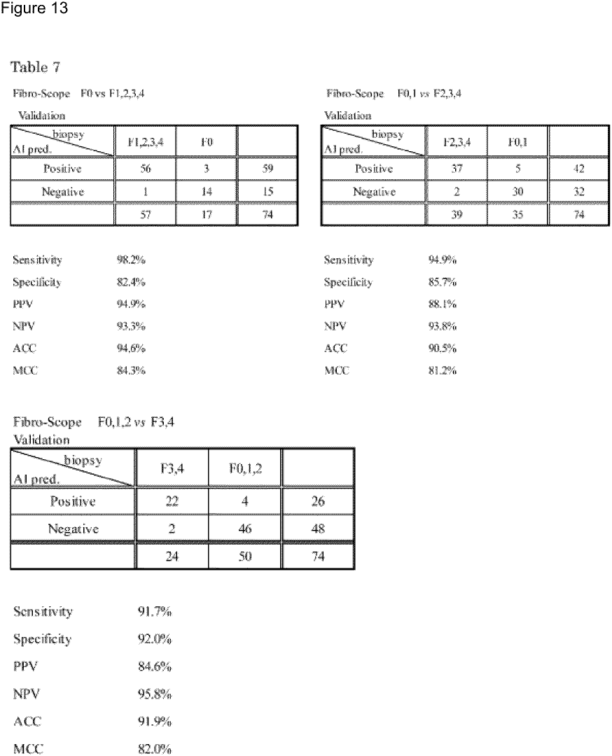

FIG. 14 is a block diagram showing a configuration of a fatty liver disease evaluation apparatus according to one embodiment of the present invention.

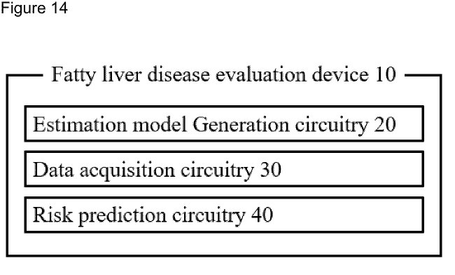

FIG. 15 shows a first page of the description according to an embodiment for carrying out the invention.

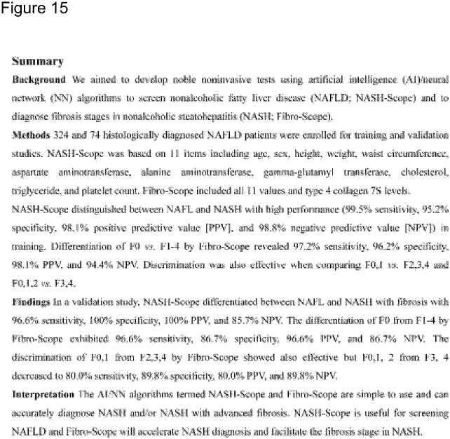

FIG. 16 shows a second page of the description according to an embodiment for carrying out the invention.

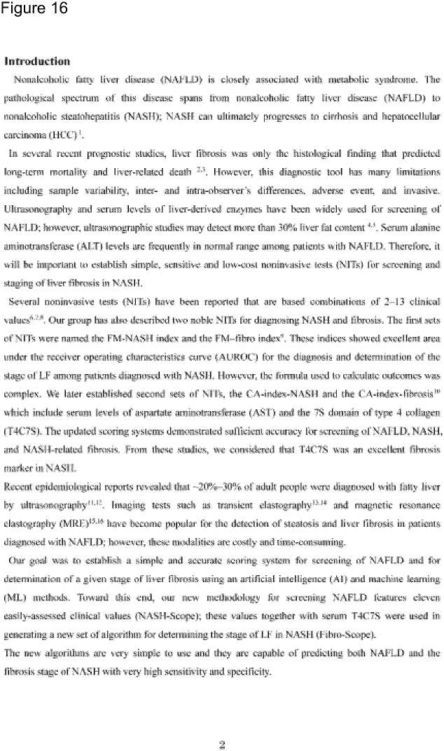

FIG. 17 shows a third page of the description according to an embodiment for carrying out the invention.

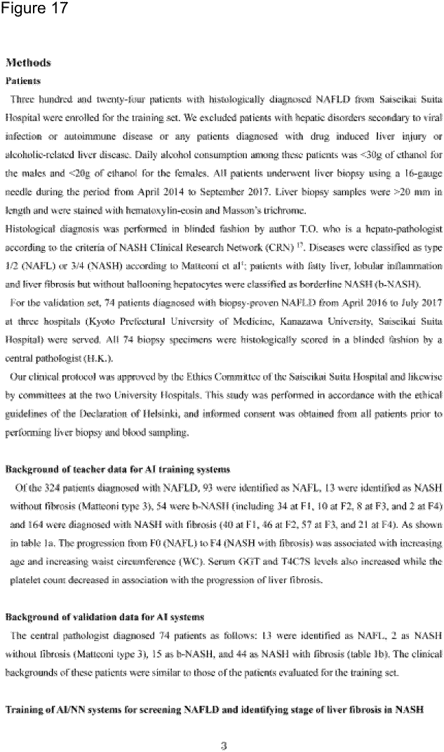

FIG. 18 shows a fourth page of the description according to an embodiment for carrying out the invention.

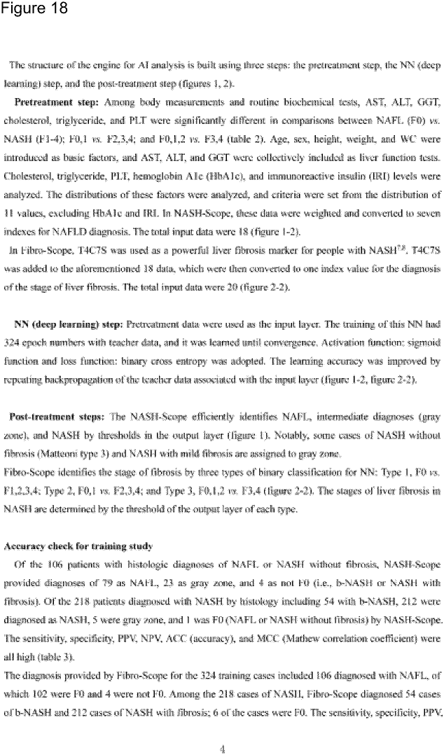

FIG. 19 shows a fifth page of the description according to an embodiment for carrying out the invention.

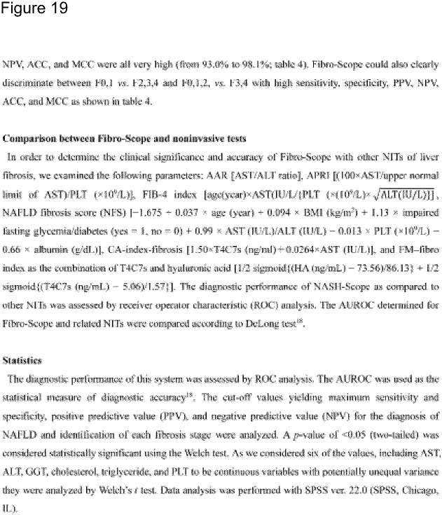

FIG. 20 shows a sixth page of the description according to an embodiment for carrying out the invention.

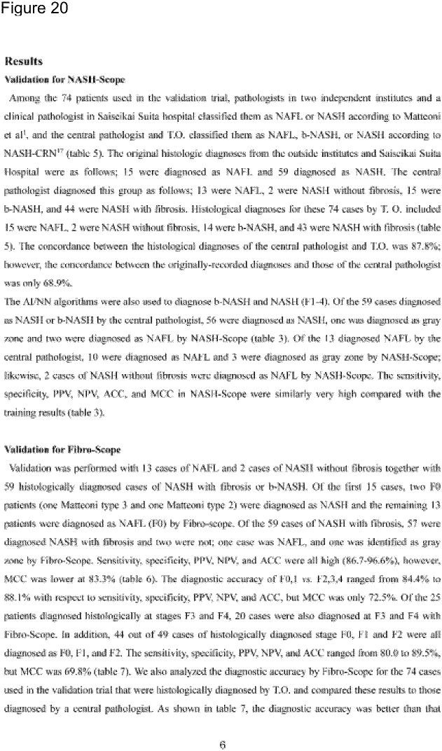

FIG. 21 shows a seventh page of the description according to an embodiment for carrying out the invention.

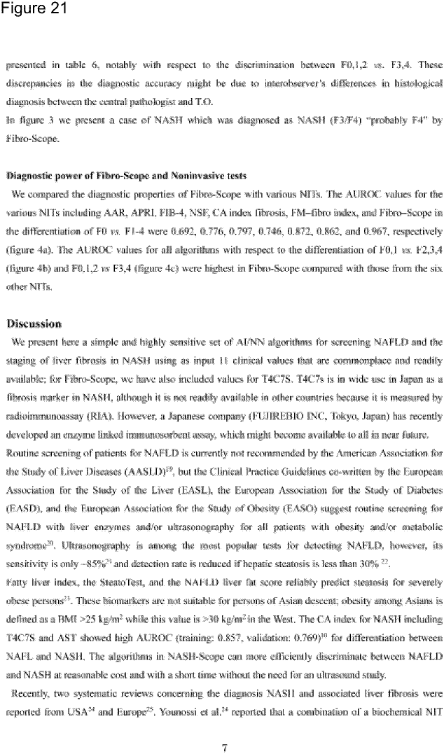

FIG. 22 shows an eighth page of the description according to an embodiment for carrying out the invention.

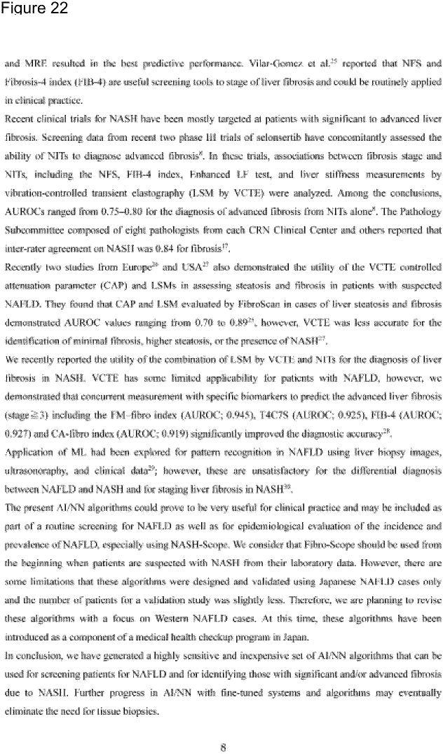

FIG. 23 shows a ninth page of the description according to an embodiment for carrying out the invention.

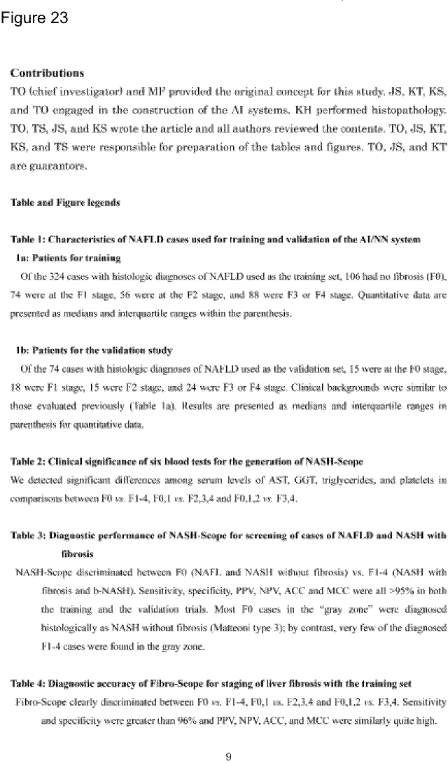

FIG. 24 shows a tenth page of the description according to an embodiment for carrying out the invention.

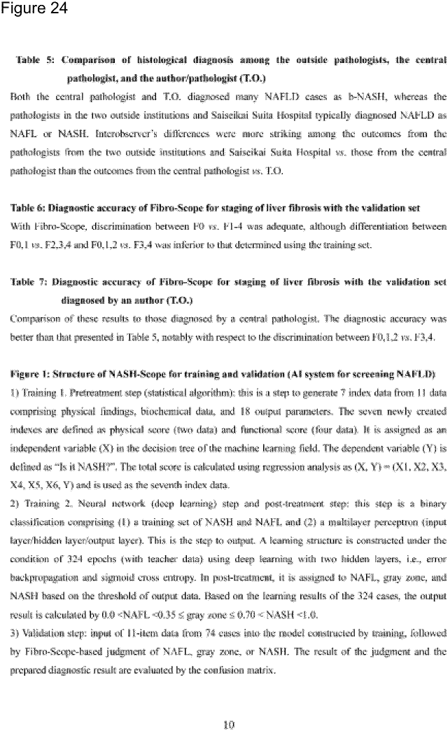

FIG. 25 shows an eleventh page of the description according to an embodiment for carrying out the invention.

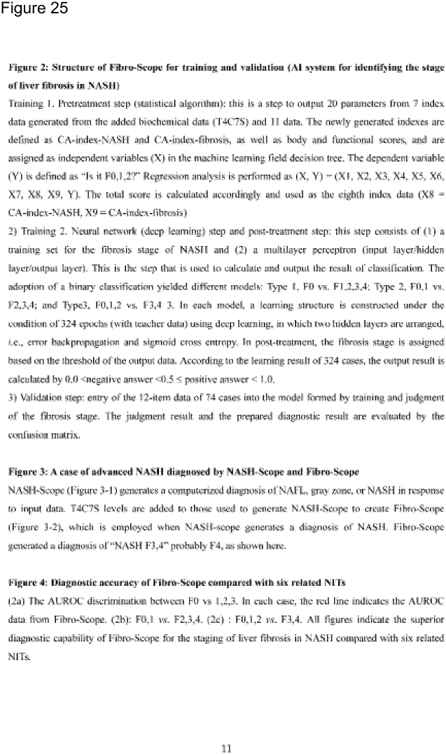

### DESCRIPTION OF EMBODIMENTS

A disease risk assessment method according to the present invention comprises:
an estimated model generation step of generating an estimated model for estimating a disease risk by machine learning using (a) an attribute data, (b) physical finding data, (c) a blood examination data, and (d) a diagnosis result data,
a data acquisition step of acquiring (a) an attribute data of a subject, (b) physical finding data of the subject, and (c) a blood examination data of the subject,
a risk level evaluation step of inputting the data acquired in the data acquisition step into the estimated model generated in the estimated model generation step and obtaining an inferred disease risk of the subject.

As the attribute data used in the estimated model generation step and the data acquisition step, it can be used such as gender, age, medical history, presence/absence of basic disease, nationality, and the like. As the body finding data used in the estimated model generation step and the data acquisition step, in addition to height and weight, blood pressure, girth, vision, hearing, body fat percentage, sitting height, head circumference, chest circumference, ultrasound and/or magnetic resonance hardness and elastography, X-ray, CT and MRI images of various areas can be used. The blood test data used in the estimated model generation step and the data acquisition step is selected from AST (also referred to as GOT), ALT (also referred to as GPT), gamma-GTP, PLT, T-Cho, TG, Alb, HDL, LDL, HbA1c, ALP, ChE, 4 type collagen (Particularly, type IV collagen. 7S), Total-AIM, Free-AIM, etc.

In the disease risk evaluation method in the present invention, the risk level related to liver diseases of the subject can be inferred. Specifically, the risk level related to liver diseases such as non-alcoholic fatty liver, liver fibrosis (hepatitis, cirrhosis), and liver cancer of the subject can be inferred.

The disease risk evaluation method, the disease risk evaluation device, and the disease risk evaluation program of the present invention are also directed to an evaluation method, an evaluation device, and an evaluation program for the non-alcoholic fatty liver. In this case, the estimated model generation step includes generating an estimated model for estimating the risk level of the non-alcoholic fatty liver by machine learning using (a) the attribute data including at least one selected from gender and age, (b) the physical finding data including at least one selected from height and weight, (c) the blood examination data including at least one selected from AST (GOT), ALT (GPT), gamma-GTP, PLT, T-Cho and TG, and (d) the diagnostic result of a doctor, the risk level evaluation step includes obtaining the risk level of the non-alcoholic fatty liver of the subject by inputting (a) the attribute data including at least one selected from gender and age of the subject, (b) the physical finding data including at least one selected from height and weight of the subject, (c) the blood examination data including at least one selected from AST (GOT), ALT (GPT), gamma-GTP, PLT, T-Cho and TG of the subject, into the estimated model in the estimated model generation step. Hereinafter, the evaluation method, the evaluation device, or the evaluation program for evaluating the degree of risk of a non-alcoholic fatty liver may be referred to as NASH-Scope.

The disease risk evaluation method, the disease risk evaluation device, and the disease risk evaluation program in the present invention are also directed to an evaluation method, an evaluation device, and an evaluation program for evaluating the hepatic fibrosis level of the liver. In this case, the estimated model generation step includes generating an estimated model for estimating the risk level of hepatic fibrosis by machine learning using (a) the attribute data including at least one selected from gender and age, (b) the physical finding data including at least one selected from height and weight, (c) the blood examination data including Type 4 collagen and at least one selected from AST (GOT), ALT (GPT), gamma-GTP, PLT, T-Cho and TG, and (d) the diagnostic result of a doctor, the risk level evaluation step includes obtaining the risk level of the hepatic fibrosis of the subject by inputting (a) the attribute data including at least one selected from gender and age of the subject, (b) the physical finding data including at least one selected from height and weight of the subject, (c) the blood examination data including Type 4 collagen and at least one selected from AST (GOT), ALT (GPT), gamma-GTP, PLT, T-Cho and TG of the subject, into the estimated model in the estimated model generation step. In other words, by further utilizing type 4 collagen (typically collagen IV 7S) as blood test data in the above-mentioned method of assessing the risk level of non-alcoholic fatty liver, it is possible to infer the subject’s liver fibrosis level. Hereinafter, the evaluation method, the evaluation device, or the evaluation program for evaluating the degree of risk of liver fibrosis may be referred to as Fibro-Scope.

The disease risk evaluation method, the disease risk evaluation device, and the disease risk evaluation program in the present invention are also directed to an evaluation method, an evaluation device, and an evaluation program for evaluating the risk level of liver cancer. In this case, the estimated model generation step includes generating an estimated model for estimating the risk level of hepatic cancer by machine learning using (a) the attribute data including at least one selected from gender and age, (b) the physical finding data including at least one selected from height and weight, (c) the blood examination data including AIM including Total-AIM and/or Free-AIM and at least one selected from AST (GOT), ALT (GPT), gamma-GTP, PLT, T-Cho and TG, and (d) the diagnostic result of a doctor, the risk level evaluation step includes obtaining the risk level of the hepatic cancer of the subject by inputting (a) the attribute data including at least one selected from gender and age of the subject, (b) the physical finding data including at least one selected from height and weight of the subject, (c) the blood examination data including AIM including Total-AIM and/or Free-AIM and at least one selected from AST (GOT), ALT (GPT), gamma-GTP, PLT, T-Cho and TG of the subject, into the estimated model in the estimated model generation step. In other words, by using AIM as blood test data in the above-mentioned method for evaluating the risk level of non-alcoholic fatty liver, the subject’s risk level for liver cancer can be inferred. Put another way, by using AIM instead of type 4 collagen as blood test data in the method for evaluating the level of liver fibrosis described above, the subject’s risk level for liver cancer can be inferred. Hereinafter, theevaluation method, the evaluation device, or the evaluation program for evaluating the risk level of liver cancer may be referred to as an HCC-Scope.

The method for evaluating the risk of fatty liver disease, the device for evaluating the risk of fatty liver disease, and the program for evaluating the risk of fatty liver disease according to one embodiment of the present invention will now be described in detail.

### Outline of the Invention

The present invention is described in accordance with specific embodiments. In the following description, NASH-Scope and Fibro-Scope is a kind of a method for evaluating the risk of fatty liver disease, a device for evaluating the risk of fatty liver disease, and a program for evaluating the risk of fatty liver disease according to the present invention.

The present specification provides noble noninvasive tests using artificial intelligence (AI)/neural network (NN) algorithms to screen nonalcoholic fatty liver disease (NAFLD; NASH-Scope) and to diagnose fibrosis stages in nonalcoholic steatohepatitis (NASH; Fibro-Scope).

The validity of the NASH-Scope and Fibro-Scope, which realize the fatty liver disease risk assessment method, fatty liver disease risk assessment device, and fatty liver disease risk assessment program of the present invention, were validated and studied by the following methods. In the tests, 324 and 74 histologically diagnosed NAFLD patients were enrolled for training and validation studies. NASH-Scope was based on 11 items including age, sex, height, weight, waist circumference, aspartate aminotransferase, alanine aminotransferase, gamma-glutamyl transferase, cholesterol, triglyceride, and platelet count. Fibro-Scope included all 11 values and type 4 collagen 7S levels.

NASH-Scope distinguished between NAFL and NASH with high performance (99.5% sensitivity, 95.2% specificity, 98.1% positive predictive value [PPV], and 98.8% negative predictive value [NPV]) in training. Differentiation of F0 vs. F1-4 by Fibro-Scope revealed 97.2% sensitivity, 96.2% specificity, 98.1% PPV, and 94.4% NPV. Discrimination was also effective when comparing F0,1 vs. F2,3,4 and F0,1,2 vs. F3,4. (F1; mild fibrosis, F2; moderate fibrosis, F3; high fibrosis, F4; cirrhosis)

In a validation study, NASH-Scope differentiated between NAFL and NASH with fibrosis with 96.6% sensitivity, 100% specificity, 100% PPV, and 85.7% NPV. The differentiation of F0 from F1-4 by Fibro-Scope exhibited 96.6% sensitivity, 86.7% specificity, 96.6% PPV, and 86.7% NPV. The discrimination of F0,1 from F2,3,4 by Fibro-Scope showed also effective but F0,1, 2 from F3, 4 decreased to 80.0% sensitivity, 89.8% specificity, 80.0% PPV, and 89.8% NPV. The AI/NN algorithms termed NASH-Scope and Fibro-Scope are simple to use and can accurately diagnose NASH and/or NASH with advanced fibrosis. NASH-Scope is useful for screening NAFLD and Fibro-Scope will accelerate NASH diagnosis and facilitate the fibrosis stage in NASH.

Nonalcoholic fatty liver disease (NAFLD) is closely associated with metabolic syndrome. The pathological spectrum of this disease spans from nonalcoholic fatty liver disease (NAFLD) to nonalcoholic steatohepatitis (NASH); NASH can ultimately progresses to cirrhosis and hepatocellular carcinoma (HCC) .

### Summary

In several recent prognostic studies, liver fibrosis was only the histological finding that predicted long-term mortality and liver-related death. However, this diagnostic tool has many limitations including sample variability, inter- and intra-observer’s differences, adverse event, and invasive. Ultrasonography and serum levels of liver-derived enzymes have been widely used for screening of NAFLD; however, ultrasonographic studies may detect more than 30% liver fat content. Serum alanine aminotransferase (ALT) levels are frequently in normal range among patients with NAFLD. Therefore, it will be important to establish simple, sensitive and low-cost noninvasive tests (NITs) for screening and staging of liver fibrosis in NASH. Several noninvasive tests (NITs) have been reported that are based combinations of 2-13 clinical values. Our group has also described two noble NITs for diagnosing NASH and fibrosis. The first sets of NITs were named the FM-NASH index and the FM-fibro index. These indices showed excellent area under the receiver operating characteristics curve (AUROC) for the diagnosis and determination of the stage of LF among patients diagnosed with NASH. However, the formula used to calculate outcomes was complex. We later established second sets of NITs, the CA-index-NASH and the CA-index-fibrosis which include serum levels of aspartate aminotransferase (AST) and the 7S domain of type 4 collagen (T4C7S). The updated scoring systems demonstrated sufficient accuracy for screening of NAFLD, NASH, and NASH-related fibrosis. From these studies, we considered that T4C7S was an excellent fibrosis marker in NASH. Recent epidemiological reports revealed that ~20%-30% of adult people were diagnosed with fatty liver by ultrasonography. Imaging tests such as transient elastography and magnetic resonance elastography (MRE) have become popular for the detection of steatosis and liver fibrosis in patients diagnosed with NAFLD; however, these modalities are costly and time-consuming.

Our goal was to establish a simple and accurate scoring system for screening of NAFLD and for determination of a given stage of liver fibrosis using an artificial intelligence (AI) and machine learning (ML) methods. Toward this end, our new methodology for screening NAFLD features eleven easily-assessed clinical values (NASH-Scope); these values together with serum T4C7S were used in generating a new set of algorithm for determining the stage of LF in NASH (Fibro-Scope). The new algorithms are very simple to use and they are capable of predicting both NAFLD and the fibrosis stage of NASH with very high sensitivity and specificity.

### Study

Three hundred and twenty-four patients with histologically diagnosed NAFLD from Saiseikai Suita Hospital were enrolled for the training set. We excluded patients with hepatic disorders secondary to viral infection or autoimmune disease or any patients diagnosed with drug induced liver injury or alcoholic-related liver disease. Daily alcohol consumption among these patients was <30 g of ethanol for the males and <20 g of ethanol for the females. All patients underwent liver biopsy using a 16-gauge needle during the period from April 2014 to September 2017. Liver biopsy samples were >20 mm in length and were stained with hematoxylin-eosin and Masson’s trichrome.

Histological diagnosis was performed in blinded fashion by Takeshi Okanoue who is a hepato-pathologist according to the criteria of NASH Clinical Research Network (CRN). Diseases were classified as type ½ (NAFL) or ¾ (NASH) according to Matteoni et al1; patients with fatty liver, lobular inflammation and liver fibrosis but without ballooning hepatocytes were classified as borderline NASH (b-NASH).

For the validation set, 74 patients diagnosed with biopsy-proven NAFLD from April 2016 to July 2017 at three hospitals (Kyoto Prefectural University of Medicine, Kanazawa University, Saiseikai Suita Hospital) were served. All 74 biopsy specimens were histologically scored in a blinded fashion by a central pathologist (Kenichi Harada).

The clinical protocol was approved by the Ethics Committee of the Saiseikai Suita Hospital and likewise by committees at the two University Hospitals. This study was performed in accordance with the ethical guidelines of the Declaration of Helsinki, and informed consent was obtained from all patients prior to performing liver biopsy and blood sampling.

### Background of Teacher Data for AI Training Systems

Of the 324 patients diagnosed with NAFLD, 93 were identified as NAFL, 13 were identified as NASH without fibrosis (Matteoni type 3), 54 were b-NASH (including 34 at F1, 10 at F2, 8 at F3, and 2 at F4) and 164 were diagnosed with NASH with fibrosis (40 at F1, 46 at F2, 57 at F3, and 21 at F4). As shown in table 1a. The progression from F0 (NAFL) to F4 (NASH with fibrosis) was associated with increasing age and increasing waist circumference (WC). Serum GGT and T4C7S levels also increased while the platelet count decreased in association with the progression of liver fibrosis.

### Background of Validation Data for AI Systems

The central pathologist diagnosed 74 patients as follows: 13 were identified as NAFL, 2 as NASH without fibrosis (Matteoni type 3), 15 as b-NASH, and 44 as NASH with fibrosis (table 1b). The clinical backgrounds of these patients were similar to those of the patients evaluated for the training set.

### Training of AI/NN Systems for Screening NAFLD and Identifying Stage of Liver Fibrosis in NASH

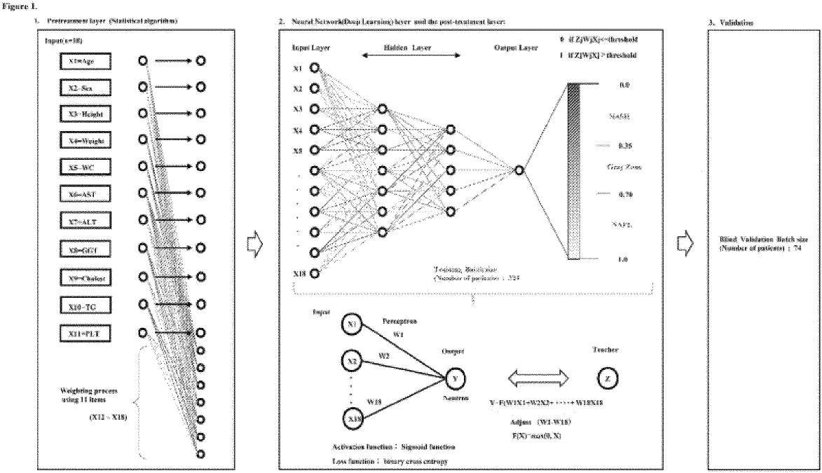

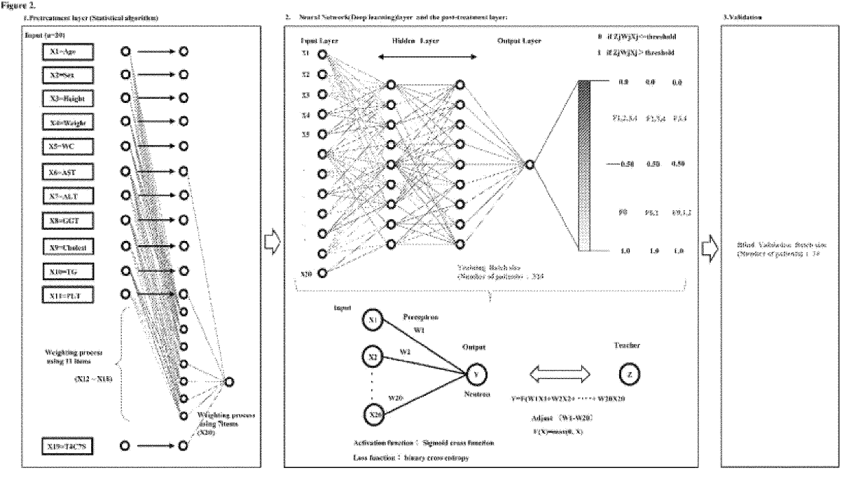

The structure of the engine for AI analysis is built using three steps: the pretreatment step, the NN (deep learning) step, and the post-treatment step (FIGS. 1, 2).

### Pretreatment Step

Among body measurements and routine biochemical tests, AST, ALT, GGT, cholesterol, triglyceride, and PLT were significantly different in comparisons between NAFL (F0) vs. NASH (F1-4); F0,1 vs. F2,3,4; and F0,1,2 vs. F3,4 (table 2). Age, sex, height, weight, and WC were introduced as basic factors, and AST, ALT, and GGT were collectively included as liver function tests. Cholesterol, triglyceride, PLT, hemoglobin A1c (HbA1c), and immunoreactive insulin (IRI) levels were analyzed. The distributions of these factors were analyzed, and criteria were set from the distribution of 11 values, excluding HbA1c and IRI. In NASH-Scope, these data were weighted and converted to seven indexes for NAFLD diagnosis. The total input data were 18 (FIGS. 1-2).

In Fibro-Scope, T4C7S was used as a powerful liver fibrosis marker for people with NASH7,8. T4C7S was added to the aforementioned 18 data, which were then converted to one index value for the diagnosis of the stage of liver fibrosis. The total input data were 20 (FIG. 2-2).

### NN (Deep Learning) Step

Pretreatment data were used as the input layer. The training of this NN had 324 epoch numbers with teacher data, and it was learned until convergence. Activation function: sigmoid function and loss function: binary cross entropy was adopted. The learning accuracy was improved by repeating backpropagation of the teacher data associated with the input layer (FIGS. 1-2, FIG. 2-2).

### Post-Treatment Steps

The NASH-Scope efficiently identifies NAFL, intermediate diagnoses (gray zone), and NASH by thresholds in the output layer (FIG. 1). Notably, some cases of NASH without fibrosis (Matteoni type 3) and NASH with mild fibrosis are assigned to gray zone.

Fibro-Scope identifies the stage of fibrosis by three types of binary classification for NN: Type 1, F0 vs. F1,2,3,4; Type 2, F0,1 vs. F2,3,4; and Type 3, F0,1,2 vs. F3,4 (FIG. 2-2). The stages of liver fibrosis in NASH are determined by the threshold of the output layer of each type.

### Accuracy Check for Training Study

Of the 106 patients with histologic diagnoses of NAFL or NASH without fibrosis, NASH-Scope provided diagnoses of 79 as NAFL, 23 as gray zone, and 4 as not F0 (i.e., b-NASH or NASH with fibrosis). Of the 218 patients diagnosed with NASH by histology including 54 with b-NASH, 212 were diagnosed as NASH, 5 were gray zone, and 1 was F0 (NAFL or NASH without fibrosis) by NASH-Scope. The sensitivity, specificity, PPV, NPV, ACC (accuracy), and MCC (Mathew correlation coefficient) were all high (table 3).

The diagnosis provided by Fibro-Scope for the 324 training cases included 106 diagnosed with NAFL, of which 102 were F0 and 4 were not F0. Among the 218 cases of NASH, Fibro-Scope diagnosed 54 cases of b-NASH and 212 cases of NASH with fibrosis; 6 of the cases were F0. The sensitivity, specificity, PPV, NPV, ACC, and MCC were all very high (from 93.0% to 98.1%; table 4). Fibro-Scope could also clearly discriminate between F0,1 vs. F2,3,4 and F0,1,2, vs. F3,4 with high sensitivity, specificity, PPV, NPV, ACC, and MCC as shown in table 4.

### Comparison Between Fibro-Scope and Noninvasive Tests

In order to determine the clinical significance and accuracy of Fibro-Scope with other NITs of liver fibrosis, we examined the following parameters: AAR[AST/ALT ratio],

$$\text{AAR} \left[\text{AST/ALT} \text{ratio}\right] \text{,}$$

$$\text{APRI} \left[\left(100 \times \text{AST} / \text{upper normal limit of AST}\right) / \text{PLT} \left(\times {10}^{9} / L\right)\right] ,$$

$$\text{FIB-4 index} \left[a g e \left(y e a r\right) \times A S T \left(I U / L / \left\{P L T \left(\times \left({10}^{9} / L\right) \times \sqrt{A L T \left(I U / L\right)}\right)\right\}\right)\right] ,$$

$$\begin{matrix}\text{NAFLD fibrosis score} \left(N F S\right) \\ \left[- 1.675 + 0.037 \times a g e \left(y e a r\right) + 0.094 \times B M I \left(k g / {m}^{2}\right) +\right) \\ 1.13 \times \text{impaired fasting glycemia/diabetes} \left(y e s = 1 , n o = 0\right) + 0.99 \times \\ A S T \left(I U / L\right) / A L T \left(I U / L\right) - 0.013 \times P L T \left(\times {10}^{9} / L\right) - 0.66 \times \text{albumin} \left(\left(g / d L\right)\right]\end{matrix}$$

$$\text{CA-index-fibrosis} \left[1.50 \times T 4 C 7 s \left(n g / m l\right) + 0.0264 \times A S T \left(I U / L\right)\right] ,$$

and FM-fibro index as the combination of T4C7s and hyaluronic acid

$$\left[1 / 2 \text{sigmoid} \left\{\left(H A \left(n g / m l\right) - 73.56\right) / 86.13\right\} + 1 / 2 \text{sigmoid} \left\{\left(T 4 C 7 s \left(n g / m l\right) - 5.06\right) / 1.57\right\}\right] .$$

The diagnostic performance of NASH-Scope as compared to other NITs was assessed by receiver operator characteristic (ROC) analysis. The AUROC determined for Fibro-Scope and related NITs were compared according to DeLong test.

### Statistics

The diagnostic performance of this system was assessed by ROC analysis. The AUROC was used as the statistical measure of diagnostic accuracy. The cut-off values yielding maximum sensitivity and specificity, positive predictive value (PPV), and negative predictive value (NPV) for the diagnosis of NAFLD and identification of each fibrosis stage were analyzed. A p-value of <0.05 (two-tailed) was considered statistically significant using the Welch test. As we considered six of the values, including AST, ALT, GGT, cholesterol, triglyceride, and PLT to be continuous variables with potentially unequal variance they were analyzed by Welch’s t test. Data analysis was performed with SPSS ver. 22.0 (SPSS, Chicago, IL).

### Results

### Validation for NASH-Scope

Among the 74 patients used in the validation trial, pathologists in two independent institutes and a clinical pathologist in Saiseikai Suita hospital classified them as NAFL or NASH according to Matteoni et al, and the central pathologist and Takeshi Okanoue classified them as NAFL, b-NASH, or NASH according to NASH-CRN (table 5). The original histologic diagnoses from the outside institutes and Saiseikai Suita Hospital were as follows; 15 were diagnosed as NAFL and 59 diagnosed as NASH. The central pathologist diagnosed this group as follows; 13 were NAFL, 2 were NASH without fibrosis, 15 were b-NASH, and 44 were NASH with fibrosis. Histological diagnoses for these 74 cases by Takeshi Okanoue included 15 were NAFL, 2 were NASH without fibrosis, 14 were b-NASH, and 43 were NASH with fibrosis (table 5). The concordance between the histological diagnoses of the central pathologist and Takeshi Okanoue was 87.8%; however, the concordance between the originally-recorded diagnoses and those of the central pathologist was only 68.9%.

The AI/NN algorithms were also used to diagnose b-NASH and NASH (F1-4). Of the 59 cases diagnosed as NASH or b-NASH by the central pathologist, 56 were diagnosed as NASH, one was diagnosed as gray zone and two were diagnosed as NAFL by NASH-Scope (table 3). Of the 13 diagnosed NAFL by the central pathologist, 10 were diagnosed as NAFL and 3 were diagnosed as gray zone by NASH-Scope; likewise, 2 cases of NASH without fibrosis were diagnosed as NAFL by NASH-Scope. The sensitivity, specificity, PPV, NPV, ACC, and MCC in NASH-Scope were similarly very high compared with the training results (table 3).

### Validation for Fibro-Scope

Validation was performed with 13 cases of NAFL and 2 cases of NASH without fibrosis together with 59 histologically diagnosed cases of NASH with fibrosis or b-NASH. Of the first 15 cases, two F0 patients (one Matteoni type 3 and one Matteoni type 2) were diagnosed as NASH and the remaining 13 patients were diagnosed as NAFL (F0) by Fibro-scope. Of the 59 cases of NASH with fibrosis, 57 were diagnosed NASH with fibrosis and two were not; one case was NAFL, and one was identified as gray zone by Fibro-Scope. Sensitivity, specificity, PPV, NPV, and ACC were all high (86.7-96.6%), however, MCC was lower at 83.3% (table 6). The diagnostic accuracy of F0,1 vs. F2,3,4 ranged from 84.4% to 88.1% with respect to sensitivity, specificity, PPV, NPV, and ACC, but MCC was only 72.5%. Of the 25 patients diagnosed histologically at stages F3 and F4, 20 cases were also diagnosed at F3 and F4 with Fibro-Scope. In addition, 44 out of 49 cases of histologically diagnosed stage F0, F1 and F2 were all diagnosed as F0, F1, and F2. The sensitivity, specificity, PPV, NPV, and ACC ranged from 80.0 to 89.5%, but MCC was 69.8% (table 7). We also analyzed the diagnostic accuracy by Fibro-Scope for the 74 cases used in the validation trial that were histologically diagnosed by Takeshi Okanoue and compared these results to those diagnosed by a central pathologist. As shown in table 7, the diagnostic accuracy was better than that presented in table 6, notably with respect to the discrimination between F0,1,2 vs. F3,4. These discrepancies in the diagnostic accuracy might be due to interobserver’s differences in histological diagnosis between the central pathologist and Takeshi Okanoue.

In FIGS. 3-1 and FIGS. 3-2, we present a case of NASH which was diagnosed as NASH (F3/F4) “probably F4” by Fibro-Scope.

### Diagnostic Power of Fibro-Scope and Noninvasive Tests

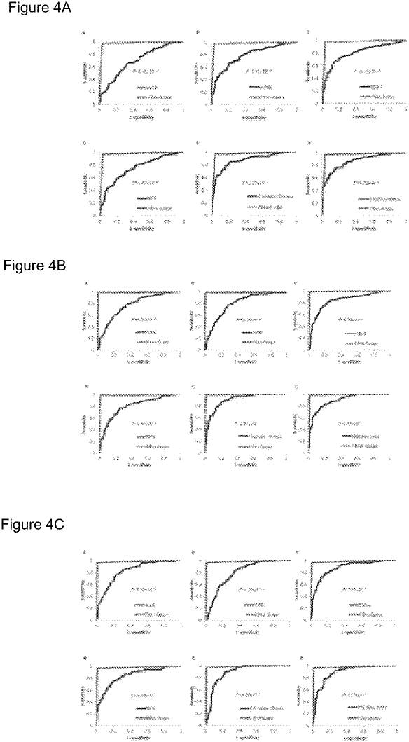

We compared the diagnostic properties of Fibro-Scope with various NITs. The AUROC values for the various NITs including AAR, APRI, FIB-4, NSF, CA index fibrosis, FM-fibro index, and Fibro-Scope in the differentiation of F0 vs. F1-4 were 0.692, 0.776, 0.797, 0.746, 0.872, 0.862, and 0.967, respectively (FIG. 4A). The AUROC values for all algorithms with respect to the differentiation of F0,1 vs. F2,3,4 (FIG. 4B) and F0,1,2 vs F3,4 (FIG. 4C) were highest in Fibro-Scope compared with those from the six other NITs.

### Discussion

We present here a simple and highly sensitive set of AI/NN algorithms for screening NAFLD and the staging of liver fibrosis in NASH using as input 11 clinical values that are commonplace and readily available; for Fibro-Scope, we have also included values for T4C7S. T4C7s is in wide use in Japan as a fibrosis marker in NASH, although it is not readily available in other countries because it is measured by radioimmunoassay (RIA). However, a Japanese company (FUJIREBIO INC, Tokyo, Japan) has recently developed an enzyme linked immunosorbent assay, which might become available to all in near future.

Routine screening of patients for NAFLD is currently not recommended by the American Association for the Study of Liver Diseases (AASLD), but the Clinical Practice Guidelines co-written by the European Association for the Study of the Liver (EASL), the European Association for the Study of Diabetes (EASD), and the European Association for the Study of Obesity (EASO) suggest routine screening for NAFLD with liver enzymes and/or ultrasonography for all patients with obesity and/or metabolic syndrome. Ultrasonography is among the most popular tests for detecting NAFLD, however, its sensitivity is only ~85% and detection rate is reduced if hepatic steatosis is less than 30%.

Fatty liver index, the SteatoTest, and the NAFLD liver fat score reliably predict steatosis for severely obese persons. These biomarkers are not suitable for persons of Asian descent; obesity among Asians is defined as a BMI >25 kg/m² while this value is >30 kg/m² in the West. The CA index for NASH including T4C7S and AST showed high AUROC (training: 0.857, validation: 0.769) for differentiation between NAFL and NASH. The algorithms in NASH-Scope can more efficiently discriminate between NAFLD and NASH at reasonable cost and with a short time without the need for an ultrasound study.

Recently, two systematic reviews concerning the diagnosis NASH and associated liver fibrosis were reported from USA and Europe. Younossi et al. reported that a combination of a biochemical NIT and MRE resulted in the best predictive performance. Vilar-Gomez et al. reported that NFS and Fibrosis-4 index (FIB-4) are useful screening tools to stage of liver fibrosis and could be routinely applied in clinical practice.

Recent clinical trials for NASH have been mostly targeted at patients with significant to advanced liver fibrosis. Screening data from recent two phase III trials of selonsertib have concomitantly assessed the ability of NITs to diagnose advanced fibrosis. In these trials, associations between fibrosis stage and NITs, including the NFS, FIB-4 index, Enhanced LF test, and liver stiffness measurements by vibration-controlled transient elastography (LSM by VCTE) were analyzed. Among the conclusions, AUROCs ranged from 0.75-0.80 for the diagnosis of advanced fibrosis from NITs alone. The Pathology Subcommittee composed of eight pathologists from each CRN Clinical Center and others reported that inter-rater agreement on NASH was 0.84 for fibrosis.

Recently two studies from Europe and USA also demonstrated the utility of the VCTE controlled attenuation parameter (CAP) and LSMs in assessing steatosis and fibrosis in patients with suspected NAFLD. They found that CAP and LSM evaluated by FibroScan in cases of liver steatosis and fibrosis demonstrated AUROC values ranging from 0.70 to 0.89, however, VCTE was less accurate for the identification of minimal fibrosis, higher steatosis, or the presence of NASH.

We recently reported the utility of the combination of LSM by VCTE and NITs for the diagnosis of liver fibrosis in NASH. VCTE has some limited applicability for patients with NAFLD, however, we demonstrated that concurrent measurement with specific biomarkers to predict the advanced liver fibrosis (stage≧3) including the FM-fibro index (AUROC; 0.945), T4C7S (AUROC; 0.925), FIB-4 (AUROC; 0.927) and CA-fibro index (AUROC; 0.919) significantly improved the diagnostic accuracy.

Application of ML had been explored for pattern recognition in NAFLD using liver biopsy images, ultrasonoraphy, and clinical data; however, these are unsatisfactory for the differential diagnosis between NAFLD and NASH and for staging liver fibrosis in NASH. The present AI/NN algorithms could prove to be very useful for clinical practice and may be included as part of a routine screening for NAFLD as well as for epidemiological evaluation of the incidence and prevalence of NAFLD, especially using NASH-Scope. We consider that Fibro-Scope should be used from the beginning when patients are suspected with NASH from their laboratory data. However, there are some limitations that these algorithms were designed and validated using Japanese NAFLD cases only and the number of patients for a validation study was slightly less. Therefore, we are planning to revise these algorithms with a focus on Western NAFLD cases. At this time, these algorithms have been introduced as a component of a medical health checkup program in Japan.

In conclusion, we have generated a highly sensitive and inexpensive set of AI/NN algorithms that can be used for screening patients for NAFLD and for identifying those with significant and/or advanced fibrosis due to NASH. Further progress in AI/NN with fine-tuned systems and algorithms may eventually eliminate the need for tissue biopsies.

The following is a more detailed explanation with reference to the figures and tables attached to this patent application.

Table 1 shows the characteristics of the NAFLD cases used for training and validation of the AI/NN system.

### 1A: Patients for Training

Of the 324 cases with histologic diagnoses of NAFLD used as the training set, 106 had no fibrosis (F0), 74 were at the F1 stage, 56 were at the F2 stage, and 88 were F3 or F4 stage. Quantitative data are presented as medians and interquartile ranges within the parenthesis.

### 1B: Patients for the Validation Study

Of the 74 cases with histologic diagnoses of NAFLD used as the validation set, 15 were at the F0 stage, 18 were F1 stage, 15 were F2 stage, and 24 were F3 or F4 stage. Clinical backgrounds were similar to those evaluated previously (Table 1a). Results are presented as medians and interquartile ranges in parenthesis for quantitative data.

Table 2 shows the clinical significance of six blood tests for the generation of NASH-Scope. We detected significant differences among serum levels of AST, GGT, triglycerides, and platelets in comparisons between F0 vs. F1-4, F0,1 vs. F2,3,4 and F0,1,2 vs. F3,4.

Table 3 shows the diagnostic performance of NASH-Scope for screening of cases of NAFLD and NASH with fibrosis. NASH-Scope discriminated between F0 (NAFL and NASH without fibrosis) vs. F1-4 (NASH with fibrosis and b-NASH). Sensitivity, specificity, PPV, NPV, ACC and MCC were all >95% in both the training and the validation trials. Most F0 cases in the “gray zone” were diagnosed histologically as NASH without fibrosis (Matteoni type 3); by contrast, very few of the diagnosed F1-4 cases were found in the gray zone.

Table 4 shows the diagnostic accuracy of Fibro-Scope for staging of liver fibrosis with the training sets. Fibro-Scope clearly discriminated between F0 vs. F1-4, F0,1 vs. F2,3,4 and F0,1,2 vs. F3,4. Sensitivity and specificity were greater than 96% and PPV, NPV, ACC, and MCC were similarly quite high.

Table 5 shows the comparison of histological diagnosis among the outside pathologists, the central pathologist, and the author/pathologist (inventor). Both the central pathologist and T.O. diagnosed many NAFLD cases as b-NASH, whereas the pathologists in the two outside institutions and Saiseikai Suita Hospital typically diagnosed NAFLD as NAFL or NASH. Interobserver’s differences were more striking among the outcomes from the pathologists from the two outside institutions and Saiseikai Suita Hospital vs. those from the central pathologist than the outcomes from the central pathologist vs. Takeshi Okanoue.

Table 6 shows the diagnostic accuracy of Fibro-Scope for staging of liver fibrosis with the validation sets. With Fibro-Scope, discrimination between F0 vs. F1-4 was adequate, although differentiation between F0,1 vs. F2,3,4 and F0,1,2 vs. F3,4 was inferior to that determined using the training set.

Table 7 shows the diagnostic accuracy of Fibro-Scope for staging of liver fibrosis with the validation set diagnosed by an author (Takeshi Okanoue). Comparison of these results to those diagnosed by a central pathologist. The diagnostic accuracy was better than that presented in Table 5, notably with respect to the discrimination between F0,1,2 vs. F3,4.

FIG. 1 illustrates the structure of NASH-Scope for training and validation (AI system for screening NAFLD).

1) Training 1. Pretreatment step (statistical algorithm): this is a step to generate 7 index data from 11 data comprising physical findings, biochemical data, and 18 output parameters. The seven newly created indexes are defined as physical score (two data) and functional score (four data). It is assigned as an independent variable (X) in the decision tree of the machine learning field. The dependent variable (Y) is defined as “Is it NASH?”. The total score is calculated using regression analysis as (X, Y) = (X1, X2, X3, X4, X5, X6, Y) and is used as the seventh index data.

2) Training 2. Neural network (deep learning) step and post-treatment step: this step is a binary classification comprising (1) a training set of NASH and NAFL and (2) a multilayer perceptron (input layer/hidden layer/output layer). This is the step to output. A learning structure is constructed under the condition of 324 epochs (with teacher data) using deep learning with two hidden layers, i.e., error backpropagation and sigmoid cross entropy. In post-treatment, it is assigned to NAFL, gray zone, and NASH based on the threshold of output data. Based on the learning results of the 324 cases, the output result is calculated by 0.0 <NAFL <0.35 ≤ gray zone ≤ 0.70 < NASH <1.0.

3) Validation step: input of 11-item data from 74 cases into the model constructed by training, followed by Fibro-Scope-based judgment of NAFL, gray zone, or NASH. The result of the judgment and the prepared diagnostic result are evaluated by the confusion matrix.

FIG. 2 is a diagram illustrating the Structure of Fibro-Scope for training and validation (AI system for identifying the stage of liver fibrosis in NASH).

1) Training 1. Pretreatment step (statistical algorithm): this is a step to output 20 parameters from 7 index data generated from the added biochemical data (T4C7S) and 11 data. The newly generated indexes are defined as CA-index-NASH and CA-index-fibrosis, as well as body and functional scores, and are assigned as independent variables (X) in the machine learning field decision tree. The dependent variable (Y) is defined as “Is it F0,1,2?” Regression analysis is performed as (X, Y) = (X1, X2, X3, X4, X5, X6, X7, X8, X9, Y). The total score is calculated accordingly and used as the eighth index data (X8 = CA-index-NASH, X9 = CA-index-fibrosis).

2) Training 2. Neural network (deep learning) step and post-treatment step: this step consists of (1) a training set for the fibrosis stage of NASH and (2) a multilayer perceptron (input layer/hidden layer/output layer). This is the step that is used to calculate and output the result of classification. The adoption of a binary classification yielded different models: Type 1, F0 vs. F1,2,3,4; Type 2, F0,1 vs. F2,3,4; and Type3, F0,1,2 vs. F3,4 3. In each model, a learning structure is constructed under the condition of 324 epochs (with teacher data) using deep learning, in which two hidden layers are arranged, i.e., error backpropagation and sigmoid cross entropy. In post-treatment, the fibrosis stage is assigned based on the threshold of the output data. According to the learning result of 324 cases, the output result is calculated by 0.0 <negative answer <0.5 ≤ positive answer < 1.0.

3) Validation step: entry of the 12-item data of 74 cases into the model formed by training and judgment of the fibrosis stage. The judgment result and the prepared diagnostic result are evaluated by the confusion matrix.

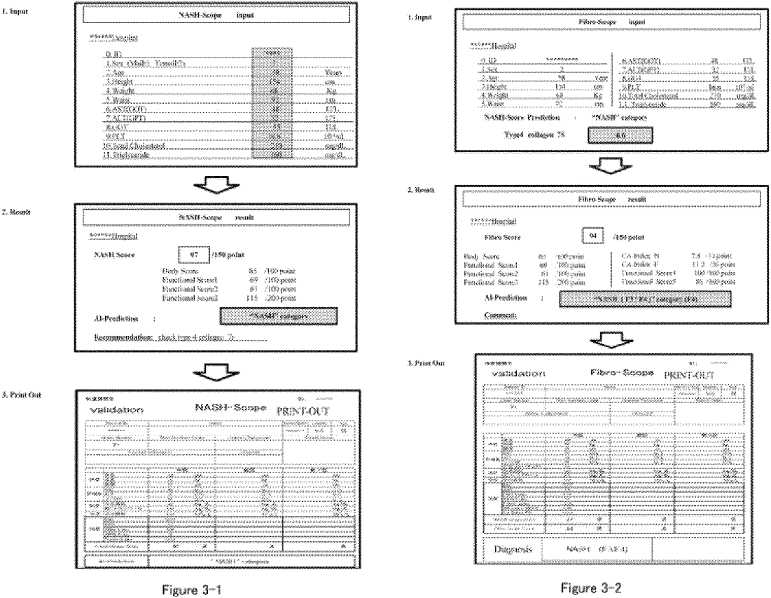

FIGS. 3-1 and FIGS. 3-2 show cases of advanced NASH diagnosed by NASH-Scope and Fibro-Scope. NASH-Scope (FIGS. 3-1) generates a computerized diagnosis of NAFL, gray zone, or NASH in response to input data. T4C7S levels are added to those used to generate NASH-Scope to create Fibro-Scope (FIGS. 3-2), which is employed when NASH-scope generates a diagnosis of NASH. Fibro-Scope generated a diagnosis of “NASH F3,4” probably F4, as shown here.

FIG. 4A, FIG. 4B and FIG. 4C show the diagnostic accuracy of Fibro-Scope compared with six related NITs. (2a) The AUROC discrimination between F0 vs 1,2,3. In each case, the red line indicates the AUROC data from Fibro-Scope. (2b): F0,1 vs. F2,3,4. (2c) : F0,1,2 vs. F3,4. All figures indicate the superior diagnostic capability of Fibro-Scope for the staging of liver fibrosis in NASH compared with six related NITs.

### The Configuration of the Fatty Liver Disease Evaluation Apparatus

The configuration of the fatty liver disease evaluation device 10 is described in this section. The fatty liver disease evaluation device 10 includes an estimation model generation circuitry 20, a data acquisition circuitry 30, and a risk level evaluation circuitry 40. The fatty liver disease evaluation device 10 is constructed by installing a program for implementing these configurations in a computer. The fatty liver disease evaluation device 10 can be used by directly operating the computer or accessing the computer through a network such as an Internet or an Intranet.

The estimation model generation circuitry 20 generates, by machine learning, an estimation model for estimating the risk level of fatty liver disease by using, as a learning model, (a) attribute data including gender and age, (b) body observation data related to body findings including height and weight, (c) biochemical data including AST (GOT), ALT (GPT), gamma GTP, PLT, T-Cho, TG, and (d) diagnostic results from a doctor.

The data acquisition circuitry 30 acquires (a) attribute data including sex and age of a subject, (b) body observation data related to physical findings including height and weight, and (c) biochemical data including AST (GOT), ALT (GPT), gamma GTP, PLT, T-Cho, TG.

The risk level prediction circuitry 40 inputs the data acquired in the data acquisition circuit to by the estimation model generated in the estimation model generation step and predicts the risk level of the fatty liver disease of the subject.

In addition to the biochemical test data, the fatty liver disease risk evaluation device 10 can generate an estimation model for estimating the risk level of the fatty liver disease by machine learning using, in addition to the biochemical test data, body finding data such as gender, age, body finding data such as height, body weight, etc. and the diagnostic result of the doctor as a learning model. In addition, the fatty liver disease risk evaluation device 10 acquires not only the biochemical test data of the subject but also body finding data such as sex, age, etc. and body finding data such as height, weight, etc. Further, the fatty liver disease risk evaluation device 10 evaluates the risk level of the fatty liver disease by the risk degree prediction unit 40 based on the estimation model generated by the estimation model generation unit 20 and the data of the subject acquired by the data acquisition unit 30. The fatty liver disease risk assessment device 10 can implement a method for predicting the risk level of a fatty liver disease of a subject by taking into account body observation data such as sex, age, etc. height, body weight, etc. by the risk degree prediction unit 40.Therefore, according to the fatty liver disease risk assessment apparatus 10, the risk of fatty liver disease can be more accurately evaluated compared to prior art methods.

The present invention is not limited to what has been shown as the embodiment, modification, etc. described above, and may be other embodiments from the teachings and spirit thereof without departing from the scope of the claims. The components of the embodiments described above may be optionally selected and combined. Any component of an embodiment or any component described in means for solving the invention or any component described in means for solving the invention may optionally be combined in any combination. These have the intention of acquiring rights in the correction or division application or the like of the present application.

### INDUSTRIAL APPLICABILITY

A disease risk assessment method, a disease risk assessment device, and a disease risk assessment program, such as fatty liver disease, capable of more accurately assessing a disease risk, such as a fatty liver disease, of the present invention are suitable for use in a method, an assessment device, and a program for the assessment device that assess the risk of various diseases.

1. A disease risk evaluation method comprising;
an estimated model generation step of generating an estimated model for estimating a disease risk by machine learning using (a) an attribute data, (b) physical finding data, (c) a blood examination data, and (d) a diagnosis result data,
a data acquisition step of acquiring (a) an attribute data of a subject, (b) physical finding data of the subject, and (c) a blood examination data of the subject,
a risk level evaluation step of inputting the data acquired in the data acquisition step into the estimated model generated in the estimated model generation step, and obtaining an inferred disease risk of the subject.

2. The disease risk evaluation method according to claim 1, wherein the inferred disease risk is the risk level for liver disease of the subject.

3. The disease risk evaluation method according to claim 2, wherein
the estimated model generation step includes generating an estimated model for estimating the risk level of the non-alcoholic fatty liver by machine learning using (a) the attribute data including at least one selected from gender and age, (b) the physical finding data including at least one selected from height and weight, (c) the blood examination data including at least one selected from AST (GOT), ALT (GPT), gamma-GTP, PLT, T-Cho and TG, and (d) the diagnostic result of a doctor,
the risk level evaluation step includes obtaining the risk level of the non-alcoholic fatty liver of the subject by inputting (a) the attribute data including at least one selected from gender and age of the subject, (b) the physical finding data including at least one selected from height and weight of the subject, (c) the blood examination data including at least one selected from AST (GOT), ALT (GPT), gamma-GTP, PLT, T-Cho and TG of the subject, into the estimated model in the estimated model generation step.

4. The disease risk evaluation method according to claim 2, wherein
the estimated model generation step includes generating an estimated model for estimating the risk level of hepatic fibrosis by machine learning using (a) the attribute data including at least one selected from gender and age, (b) the physical finding data including at least one selected from height and weight, (c) the blood examination data including Type 4 collagen and at least one selected from AST (GOT), ALT (GPT), gamma-GTP, PLT, T-Cho and TG, and (d) the diagnostic result of a doctor,
the risk level evaluation step includes obtaining the risk level of the hepatic fibrosis of the subject by inputting (a) the attribute data including at least one selected from gender and age of the subject, (b) the physical finding data including at least one selected from height and weight of the subject, (c) the blood examination data including Type 4 collagen and at least one selected from AST (GOT), ALT (GPT), gamma-GTP, PLT, T-Cho and TG of the subject, into the estimated model in the estimated model generation step.

5. The disease risk evaluation method according to claim 2, wherein
the estimated model generation step includes generating an estimated model for estimating the risk level of hepatic cancer by machine learning using (a) the attribute data including at least one selected from gender and age, (b) the physical finding data including at least one selected from height and weight, (c) the blood examination data including AIM and at least one selected from AST (GOT), ALT (GPT), gamma-GTP, PLT, T-Cho and TG, and (d) the diagnostic result of a doctor,
the risk level evaluation step includes obtaining the risk level of the hepatic cancer of the subject by inputting (a) the attribute data including at least one selected from gender and age of the subject, (b) the physical finding data including at least one selected from height and weight of the subject, (c) the blood examination data including AIM and at least one selected from AST (GOT), ALT (GPT), gamma-GTP, PLT, T-Cho and TG of the subject, into the estimated model in the estimated model generation step.

6. A disease risk evaluation device comprising;
an estimated model generation circuitry generating an estimated model for estimating a disease risk by machine learning using (a) an attribute data, (b) physical finding data, (c) a blood examination data, and (d) a diagnosis result data,
a data acquisition circuitry acquiring (a) an attribute data of a subject, (b) physical finding data of the subject, and (c) a blood examination data of the subject,
a risk level evaluation circuitry inputting the data acquired in the data acquisition step into the estimated model generated in the estimated model generation step, and obtaining an inferred disease risk of the subject.

7. A disease risk evaluation program for causing a computer to implement the function of the disease risk evaluation device in claim 6.
